(19) United States
(12) Plant Patent Application Publication
(10) Pub. No.: US 2006/0015977 P1
(43) Pub. Date: Jan. 19, 2006

(54) Juncus plant named 'NICHgul1'

(50) JUNCUS EFFUSUS; JUNCUS

(76) Inventor: Lisbeth Rostboell, Aarslev (DK)

Correspondence Address: Lisbeth Rostboell, Kirstinebjergvej 24, Aarslev, 5792, DK

(21) Appl. No.: 10/894,042

(22) Filed: Jul. 19, 2004

(51) Int. Cl. A01H 5/00

(52) U.S. Cl. PLT/373

(57) ABSTRACT

A new Juncus effusus plant which has abundant, green long lasting, yellow striped and attractive screwed stems. The variety successfully propagates from tissue and is suitable for year round production in commercial glass houses as an ornamental pot plant. This new and distinct variety has shown to be uniform and stable in the resulting generations from asexual propagation.

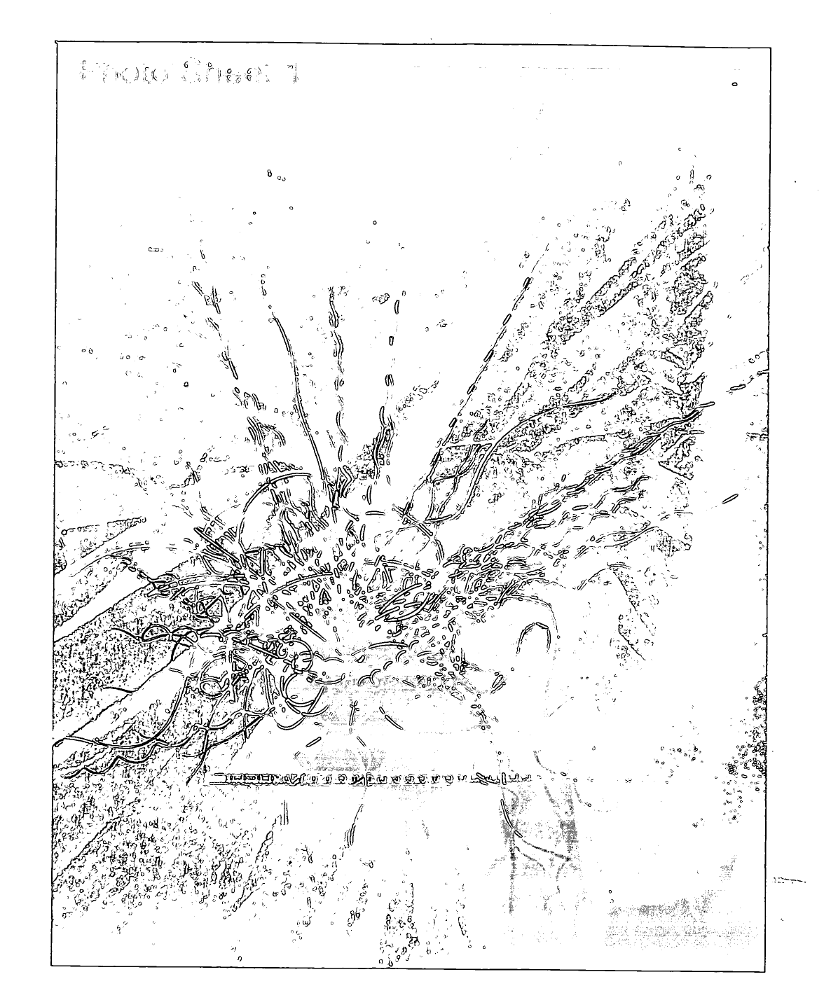

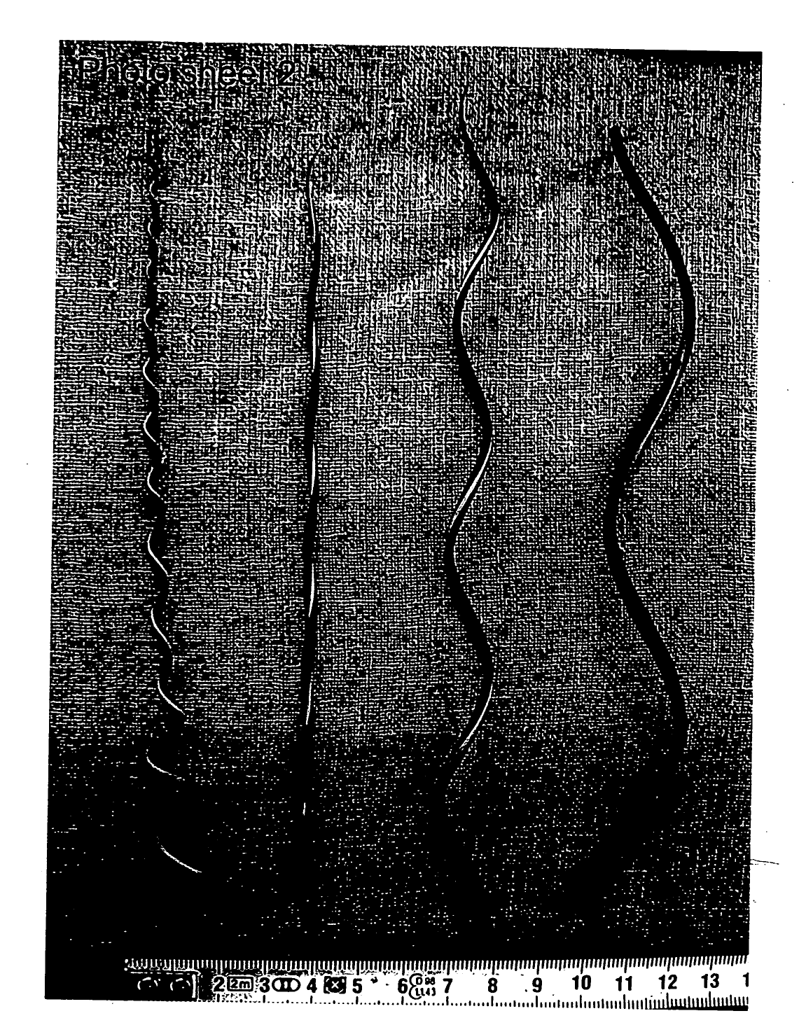

### BACKGROUND OF THE INVENTION

The present invention constitutes a new and distinct variety of Juncus effuses plant, which was discovered and selected by the Inventor, in controlled environment in AArslev, Denmark. The new plant was selected June 2003 in an unnamed selection of Juncus effusus ‘Unicorn’ (not patent in the US), also discovered and selected by the same Inventor. Out of these plants one plant was selected, as the new variety and named ‘NICHgul1’. The new plant may be distinguished from its parent, by the following combination of characteristics:

1. The parent has dark green stems.

2. ‘NICHgul1’ has dark green stems with Yellow stripes.

### BRIEF SUMMARY OF THE INVENTION

Initial asexual reproduction of ‘NICHgul1’ by tissue was first done in AArslev, Denmark. The reproduction was conducted in controlled greenhouse environments. Have here proven that the foregoing and all after characteristics and distinctions to come true to form and are established in succeeding propagations. ‘NICHgul1’ is a low and compact Juncus effusus plant with medium vigor.

The objective of the selection of this Juncus effusus variety for commercial greenhouse culture was to create a new and distinct variety with:

1. Uniform and abundant foliage with good keepability;

2. Attractive long lasting stems and compact growth, and stems with yellow stripes,

3. Year round cultivation under glasshouse conditions;

4. Suitability for production from tissue.

This combination of qualities was not present in previously available commercial cultivars of this type and distinguish ‘NICHgul1’ from all other varieties of which we are aware. The plant from the selection were planted and propagated in a controlled environment and evaluations were conducted on the resulting plants. ‘NICHgul1’ was selected by, Lisbeth Rostboell, in her development program in AArslev, Denmark.

### BRIEF DESCRIPTIONS OF THE DRAWINGS

The accompanying color illustrations show as true as is reasonably to obtain in color photographs of this type, the typical characteristics of the plant, leaves, stems of ‘NICHgul1’. Specifically illustrated in:

### Photo Sheet # 1:

1 Entire plant

### Photo Sheet # 2:

2 Young leaves entire part

3 Young leaves top part

4 Mature leaves top part

5 Mature leaves bottom part

### DETAILED BOTANICAL DESCRIPTION OF THE VARIETY

The following is a detailed description of the Juncus effuses ‘NICHgul1’. The following observations, measurements, values and comparisons describe plants grown in glass houses in AArslev, Denmark. The age of the observed plants where 11 to 13 weeks after propagation by tissue, and produced as an ornamental foliage pot plants in container of 10 centimeter in diameter. Color references are made using The Royal Horticultural Society (London, England) Colour Chart, 1995, except where common terms of color are used.
Classification:
   Botanical.—Juncus effesus.
   Commercial.—Corkscrew rush.
Plant:
   Plant growth.—Moderately vigorous. Grows compact in clumps erect or slightly arching. When grown as 10 cm pot plant, the average height of the plant itself is 18 to 20 cm, and average width is 20 cm. When grown as a 15 cm pot plant, the average height of the plant itself is 22 to 27 cm, and average width is 30 cm. Production time is generally 11 to 13 weeks depending on average temperature, light level, and cultural practices.
Stem:
   Shape, rigidly sharp-pointed, cylindrical.—Twisted left screwed.
   Color.—Young stem: Yellow-Green Group 138 A, with Stripes of Yellow-Green Group 1C at the base, to Yellow-Green Group 137 A, with stripes of Yellow-Green Group 1A at the top. Older stem: Yellow-Green Group 137 B at the base, with Stripes of Yellow-Green Group 1C at the base, To Yellow-Green Group 137A at the top. with stripes of Yellow Group 11B at the top.
   Surface.—Young wood: Smooth and slightly striated length wise, some young stems with accordion folds cross wise. Older wood: Smooth and slightly striated length wise.
   Stem diameter.—2-3 mm.
   Numbers of internodes.—none.
Plant foliage: Leaves are reddish-Green sheaths at the bottom of the stems.
   Leaf size.—Medium 30-65 mm(l). Sheath is 2-3 mm in diameter.
   Color.—Yellow-Green Group 137A-B, at the ridge, and Yellow-Green Group 1C towards the Edge with intonations of Grey-Orange Group 165B.
   Surface.—Smooth.
Inflorescense:
   Blooming habit.—No flowering have been observed.
Development:
   Vegetation.—Dense forming clumps.
   Blooming.—Not observed.
   Aptitude to bear fruit.—Not observed.
   Resistance to diseases.—No desieases have been observed under normal growing conditions in AArslev, Denmark. Hips/seeds has not been observed.
   Winter hardiness & drought/heat tolerance.—Due to the fact, that this variety is a potted ornamental plant, developed for one time use only (indoor houseplant), the plant is not tested for winter hardiness or drought/heat tolerance.

1. A new and distinct variety of Juncus effusus plant, substantially as herein illustrated and described as a distinct and novel Juncus effuses variety due to its abundant, long lasting, yellow striped stems, vigorous and compact growth, year round cultivation under glasshouse conditions, suitability for production from tissue in pots, and stems which make the variety suitable for distribution in the floral industry.
